(19) United States
(12) Patent Application Publication
(10) Pub. No.: US 2019/0206537 A1
(43) Pub. Date: Jul. 4, 2019

(54) METHOD AND SYSTEM FOR CUSTOMIZING, AGGREGATING, PRIORITIZING, AND DISPLAYING MEDICATION ADVERSE EFFECTS

(71) Applicant: Chioma Cynthia Nwaubani, Vienna, VA (US)

(72) Inventor: Chioma Cynthia Nwaubani, Vienna, VA (US)

(21) Appl. No.: 16/240,699

(22) Filed: Jan. 4, 2019

Related U.S. Application Data
(60) Provisional application No. 62/613,408, filed on Jan. 4, 2018.

(51) Int. Cl. G16H 20/10; G16H 10/60; G16H 80/00

(52) U.S. Cl. CPC G16H 20/10; G16H 50/30; G16H 80/00; G16H 10/60

(57) ABSTRACT

This invention provides a scoring and ranking system and method for a patient's medication management, using a computerized database that condenses, aggregates and organizes adverse effects in groups based on prevalence and severity while listing the offending drugs and their side effects on a color scale. This would enable clinicians to come up with certain solutions aimed at de-prescribing or eliminating the offending medications, or educating the patient on potential adverse effects.

### CROSS-REFERENCE TO RELATED APPLICATIONS

This application claims benefit under 35 U.S.C. § 119(e) of Provisional Application No. 62/613,408 filed Jan. 4, 2018, the disclosure of which is incorporated by reference in its entirety.

### FIELD

This application relates to the general field of a scoring system and method in patient's medication management, using a computerized database that condenses, aggregates and organizes adverse effects in groups based on prevalence and severity while listing the offending drugs and side effects on a color scale. The application will then suggest customized therapeutic alternatives to the offending medication based on these criteria: 1) individual patient's chronic conditions and 2) the suggested alternatives won't cause the same side effects. This would enable clinicians to come up with certain solutions aimed at de-prescribing or eliminating the offending medications, or educating the patient on potential adverse effects and then easily and efficiently replacing the offending medications with therapeutic alternatives without a tedious and extensive research, thereby saving the clinicians time.

### BACKGROUND

An indispensable aspect of an effective Medication Therapy Management (MTM) program is a software that helps to lay the foundation for successful MTM efforts. Prescription medications are effective remedies for many patients when possible side effects of each medication are well managed. However, studies have shown that, each medication has multiple side effects, some in the hundreds.

A side effect is an undesirable effect that occurs in a patient in addition to the desired therapeutic effect of a drug. Its occurrence may be unpredictable, or it may result as a part of the pharmacological action of a drug, and may vary for each individual depending on the person's disease state, age, genetic makeup, weight, gender, ethnicity and general health.

Side effects can occur when commencing, decreasing/increasing dosages, or ending a drug or medication regimen. Side effects may interfere with a prescribed treatment plan. Patients with multiple medication in their cabinets will also be exposed to multiple side effects, which in turn results in a large number of hospitalizations and readmissions every year. When side effects of a drug or medication are severe, the dosage may be adjusted or a second medication may be prescribed. Lifestyle or dietary changes may also help to minimize side effects.

Most medications have multiple adverse effects, making it very difficult and time consuming for a clinician to review and assess each potential side effect. This problem has led to very minimal or lack of side effect counselling or education, thereby leading to the prevalence of preventable medication related problems—a major driver of healthcare cost.

There is thus a need for improved methods for assisting the clinician in prioritizing, organizing, scoring and predicting the likelihood of the occurrence of medication adverse effect(s) in a patient, including an algorithm for predicting additive side effects of all the medication in a patient's cabinet.

Moreover, at present, given the prevalence or preponderance of medication side effects, it is hard for clinicians to easily visualize or prioritize which side effects to educate the patient about or which drugs to monitor or de-prescribe based on predictive side effect analysis. It would be time consuming and difficult for a clinician to go over all the myriad of side effects of all the patient's medications which could easily run into the hundreds. This issue becomes more complicated with respect to patients taking multiple medications. This information is important for several reasons, including assisting with diagnosis and counseling based on the patient's medical history and reaction to drugs.

There is thus a need to provide a method to help clinicians streamline, aggregate, score, visualize, organize and predict a patient's adverse effects in order to develop the correct intervention for the addressing medication related problems.

Also, new burdens for the healthcare professionals have been put in place under Affordable Care Act (ACA) of 2010 to improve patient outcomes through provision of comprehensive services. The ACA is expected to improve health quality and lower healthcare costs. Therein, patients are guaranteed greater access to clinical services through medical homes and a program providing medication therapy management (MTM) services as part of the collaborative patient care and coordinated home-based care for high-need patients. It is expected that more patients with chronic diseases will receive their clinical services in medication therapy management (MTM) under a new program established to improve quality of patient care and reduce overall treatment costs. This program will provide grants or contracts for clinical MTM services to collaboratively treat patients who have certain risk factors or other high-risk problems.

In addition, various states are drafting standards that meet the Federal requirements, which require the following types of information to be provided for all prescription drugs: name and description of medicine, dosage form and dosage, how to administer, duration of drug therapy, how to handle missed doses, and prescription refill information.

Accordingly, there is a need for methods for providing information which complies with the legislation, and which helps clinicians come up with an algorithm for predicting additive side effects of all the medication in a patient's cabinet for easier treatment of patients who have certain risk factors or other high-risk problems. By so doing, clinicians can serve as medication-use system experts, meet pay-for-performance measures, and prevent hospital readmissions.

Moreover, it is difficult for verbal counseling sessions to achieve this goals because patients may have multiple drugs that can result in multiple adverse effects all or part of which may reoccur soon after the session is completed. There is thus a need for a method to assist a clinician to aggregate, streamline, condense, prioritize and predict each medication's adverse effects, including an algorithm for predicting additive side effects of all the medication in a patient's cabinet.

There is a more specific need to provide a “side-effect message” that the clinician can access on the user device that compiles patient's adverse effects and scores them based on prevalence and severity using an interactive telecommunications system that helps the clinician come up with certain solutions, which is aimed at de-prescribing or eliminating the offending medications, or helping the patient implement other healthy behavioral changes based upon their medication history over a period of time.

Databases for determining side effects, such as electronic databases like FDA typically requires the user to input each drug and search for its adverse generic side effects. Not every clinician will remember to check this each time, and some side effects on some patients may not even be listed, and it is both time consuming and complex, especially when the clinicians has to check each medication in a patient's cabinet. Furthermore, it is left up to the clinician to remember to check each medication every time the patient gets a new prescription and to do so accurately. FDA database lists adverse effects but does not compile the adverse effects for each patient, and hence no algorithm to score those effects based on prevalence and severity.

There is thus a need for a system and method to enable clinicians to prioritize and predict the severity and frequency of a given side effect on a patient, and aggregating the score across multiple medications in the patient's cabinet. The application will then suggest customized therapeutic alternatives to the offending medication based on these criteria: 1) individual patient's chronic conditions and 2) the suggested alternatives won't cause the same side effects. This would enable clinicians to come up with certain solutions aimed at de-prescribing or eliminating the offending medications, or educating the patient on potential adverse effects and then easily and efficiently replacing the offending medications with therapeutic alternatives without a tedious and extensive research, thereby saving the clinicians time.

Because of differences on genetic makeup, environment, age, state of health, weight, other medications being taken, and other factors, the side effects for two patients taking the same medication may vary considerably. Therefore, a system that relies only on a comprehensive list of adverse effects as found on the FDA database, drug.com and other electronic databases may be very limited in applicability to the general population.

Systems and methods for assisting the clinician in prioritizing, organizing, scoring and predicting the likelihood of the occurrence of medication adverse effect(s) in a patient may benefit from improvements. The application will then suggest customized therapeutic alternatives to the offending medication based on these criteria: 1) individual patient's chronic conditions and 2) the suggested alternatives won't cause the same side effects. This would enable clinicians to come up with certain solutions aimed at de-prescribing or eliminating the offending medications, or educating the patient on potential adverse effects and then easily and efficiently replacing the offending medications with therapeutic alternatives without a tedious and extensive research, thereby saving the clinicians time.

### SUMMARY

In one aspect of the present invention, a method is provided. The method includes a) creating an electronic health record of a patient based on a medication list of the patient; b) storing the electronic health record of the patient into a first database; c) matching the medication list in the electronic health record with a second database of side effects data for each medication in the medication list; d) storing the side effects data for each medication in a third database, wherein the side effects data includes a frequency of occurrence for each side effect associated with each medication, wherein the side effects data includes a severity of each side effect associated with each medication; e) determining the overall side effect score based on the aggregate frequency for that side effect and the severity weight for that side effect; f) creating an electronic image that shows the side effects ranked and assigned a shade of color; and g) displaying the electronic image on a display.

Further embodiments of the disclosed system and method will become apparent from the following detailed description, the accompanying drawings and the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block diagram of the components according to one embodiment of the present invention;

FIG. 2 is a flow diagram of an exemplary method according to FIG. 1;

FIG. 3 is an electronic image of a patient profile dashboard showing the side effect scoring feature;

FIG. 4 is an electronic image of a portion of a customize side effect ranking with corresponding colors;

FIG. 5 is an expanded view of FIG. 4 showing the offending drugs for each side effect aggregate;

FIG. 6 is another view of an electronic image of a section of the customized side effect ranking with corresponding colors;

FIG. 7 is an expanded view of an electronic image showing the offending drugs and the corresponding side effects;

FIGS. 8A-8C is in three pages or sheets and illustrates the multi-medication side effect scoring according to the present invention; and

FIG. 9 is a flow diagram according to the embodiment of the present invention.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

As used herein, the terms “component” and “system” are intended to encompass hardware, software, or a combination of hardware and software. Thus, for example, a system or component may be a process, a process executing on a processor, or a processor. Additionally, a component or system may be localized on a single device or distributed across several devices.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

Drug side effects to a particular medication are listed without any scores as to severity of each on a given patient. However, research has shown that different side effects have different frequency abilities, and thus require different methods of treatment. Weighing the frequency of occurrence of each side effect using different codes can help the clinician determine the appropriate treatment plan for each patient. For example, weights can be adjusted but each weight has to be between 0 and 1, and has to reflect the probability of occurrence in each category. Such scoring algorithm is not recognized by known systems and methods.

Thus, there is a need for a method to weigh the frequency of each side effect on a given patient's medication using frequency codes that vary from 0-2 with 0 representing “more frequent” and 2 representing “incidence rare or very rare.” There is yet another need in the art to permit a clinician to make certain assumptions regarding the severity of the occurrence of each patient's side effect. For example, weighing the severity codes may lead to an assumption that a severe side effect is two times worse than a less severe side effect.

There is still a further need to provide a method that helps the clinician to determine certain treatment options based on severity codes, and also guide him/her in reaching a decision as to whether a particular side effect is more frequent or less frequent.

Similarly, there is a need for providing a formula for calculating the frequencies of side effect occurrence in a patient.

The formula provides an aggregate frequency per side effect severity. So, for each side effect, this quantity needs to be calculated twice—once for low severity (5) and once for high severity (10). Then the overall side effect score can be computed.

In databases, such as FDA database, adverse effects are generated for each drug searched in the system. However, the databases fail to provide the prevalence and severity data of the side effects for drugs in an organized or non-aggregate format. They fail to provide any formula for calculating the frequencies of side effect occurrence(s) in a patient or any method to assist the clinician, who is confronted with a patient experiencing an adverse side effect to a medication, to aggregate frequency per side effect severity.

Thus, there is a need for a formula that provides an aggregate frequency per side effect severity. So, for each side effect, the Clinician will easily calculate the severity using a simple formula. For example, for each side effect, this quantity needs to be calculated twice—once for low severity (5) and once for high severity (10). See FIG. A. Then the overall side effect score can be computed as:

Overall Side Effect Score=(Severity Weight)×(Aggregate Frequency).

Using this formula, a clinician is able to make his treatment decision based on a simple computation that takes into account the number of medications per patient and factors in the side effect frequencies per patient, and scores the severity based on this model.

For example, suppose Patient A is taking three medications #1, #2, and #3 with side effect frequencies and severities shown below.

| Medication #1 | Frequency | Severity |
|---|---|---|
| Side Effect A | 0.85 | 5 |
| Side Effect B | 0.25 | 5 |
| Side Effect C | 0.25 | 10 |

| Medication #2 | Frequency | Severity |
|---|---|---|
| Side Effect A | 0.25 | 10 |
| Side Effect B | 0.25 | 5 |
| Side Effect C | 0.25 | 5 |

| Medication #3 | Frequency | Severity |
|---|---|---|
| Side Effect A | 0.5 | 10 |
| Side Effect B | 0.5 | 5 |
| Side Effect C | 0.85 | 10 |

For known systems and methods, as with the lack of any formula for calculating side effect scores, there is no laid down formula or steps to calculate aggregate frequencies per side effect per severity level. Therefore, the known systems and methods do not give the clinician the opportunity to aggregate the frequencies per side effect per severity level. While these inadequacies continue to result in reoccurrence of side effects, which may be due to a combination of other factors, including the fact that the initial treatment did not factor in side effects resulting from the use of other medications. It may also result in a failure on the part of the clinician to understand or follow a particular treatment pattern, or lack of follow-up care, among others. Thus, these systems and methods might continue to increase readmissions significantly, increase cost and utilization in patients with adverse side effects. There is thus a need for a simple formula or method for calculating side effect scores and aggregate frequencies per side effect per severity level.

Further, in some situations, a patient may have various side effects from a particular drug use or from different drugs in their cabinet, making determination of the aggregate frequencies of side effect occurrence in that patient extremely difficult to calculate to easily come up with overall side effect score.

One approach to resolving this problem is to come up with a simple formula to assist the clinician to aggregate the frequencies of side effect occurrences and work out the expected overall side Score by simple multiplications. For example:

| Side Effect | Overall Side Effect Score |
|---|---|
| Side Effect A | 5 * 0.85 + 10 * 0.4375 = 8.625 |
| Side Effect B | 5 * 0.6875 + 10 * 0 = 3.4375 |
| Side Effect C | 5 * 0.75 + 10 * 0.25 = 6.25 |

Accordingly, there is a need for a set formula that will help clinicians generate a Score for additive side effect suffered by any patient.

Moreover, to attend to situations wherein a plurality of side effects are prevalent in a patient, there is a need for a simple mathematical formula having an ability to provide a methodical treatment by the Clinician, while saving valuable time and costs.

There are systems and methods that require the patient or the clinician to use certain programs in a database to determine the adverse side effect of each medication in their cabinet. However, these systems and methods fail to provide any method or devices meeting the above noted and long felt needs. The present invention thus provides comprehensive features and methods which meet the above described needs, overcome these deficiencies, and can best monitor and improve health care compliance and provide increased acceptance of medication adverse effects predictors combined with scoring algorithm by clinicians.

This invention addresses the above-mentioned problems. One embodiment of this invention provides a scoring and ranking system and method for a patient's medication management, using a computerized database that condenses, aggregates, ranks and organizes adverse effects in groups based on prevalence and severity while listing the offending drugs and their side effects on a color scale. This would enable clinicians to come up with certain solutions aimed at de-prescribing or eliminating the offending medications, or educating the patient on potential adverse effects in a timely manner.

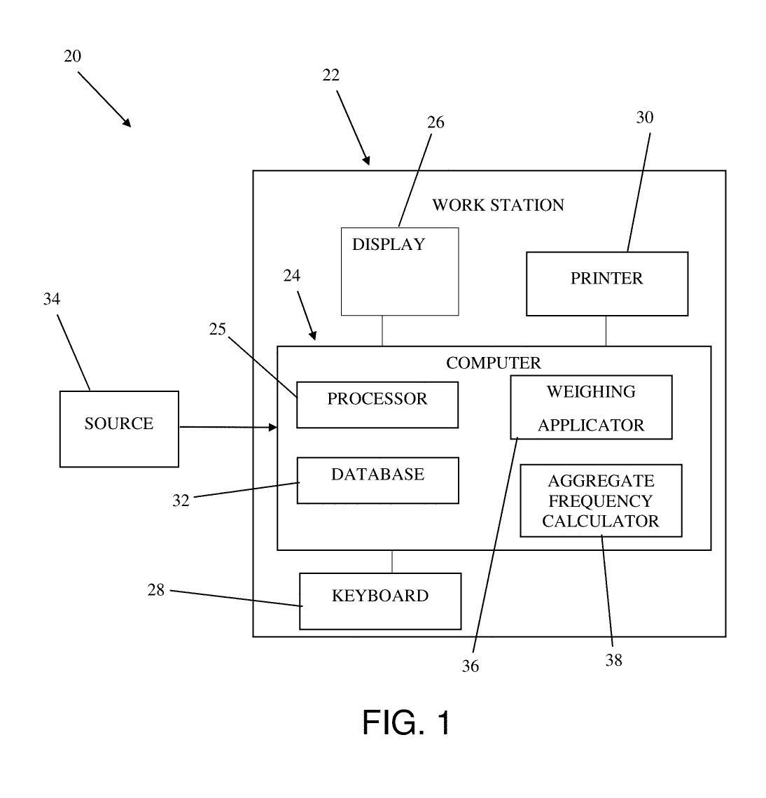

FIG. 1 shows a block diagram of the components of the system 20 for organizing, prioritizing, and predicting the probability of medication adverse effects occurring using a scoring model analysis and algorithm based on prevalence and severity. The system includes a work station 22. The work station 22 may include a computer 24, a display 26, a keyboard 28, and a printer 30. The computer 24 may have databases 32 that store information and a processor 25. The system 20 includes sources 34 for containing data of the patient's medication list or record. The sources may include data from a pharmacy claims database, hospital records, long term care facilities, or other suitable sources of data from the patient's medication list or record. The source 34 is in operative communication with the computer 24 via wire or wireless connection and may send data of the patient's medication list or record to a database 32 in the computer. The computer 24 may include a weighing applicator 36 for determining and assigning the weight for the severity of each side effect. The computer 24 may include an aggregate frequency calculator 38 for calculating the aggregate frequency of the frequencies of the each side effect for all of the medications.

The sources 34 that contain data of the patient's mediation list or record and the computer may communicate with a server system via the internet over a network. The network may include any one or combination of multiple different types of networks, such as cable networks, local area networks, personal area networks, wide area networks, the Internet, wireless networks, ad hoc networks, mesh networks, and/or the like. Alternatively or in addition, mobile devices may be in operative communication with the system to be used in combination with the computer or as a standalone devices to perform the steps.

The mobile device may be any computing device small enough to hold and operate in the hand. The mobile device may comprise a display having LCD flat screen interface that provides a touchscreen interface with digital buttons and keyboard, and/or physical buttons along with a physical keyboard. The mobile device may connect to the internet and interconnect with other devices such as car entertainment systems or headsets via Wi-Fi, Bluetooth, cellular networks or near field communication (NFC). The mobile device may be a cell phone, smart phone, tablet, PDA, laptop, notebook or other suitable portable or mobile device. The mobile device includes one or more processors and a memory.

This system 20 discloses a number of devices that use electronic technology to help the clinician determine the side effects of the medication in the patient's cabinet. These devices generally can be classified as (1) electronic scoring algorithm; (2) medication adverse effects predictors combined with scoring algorithm, and (3) fixtures for predicting medication side effects which are combined with scoring algorithm.

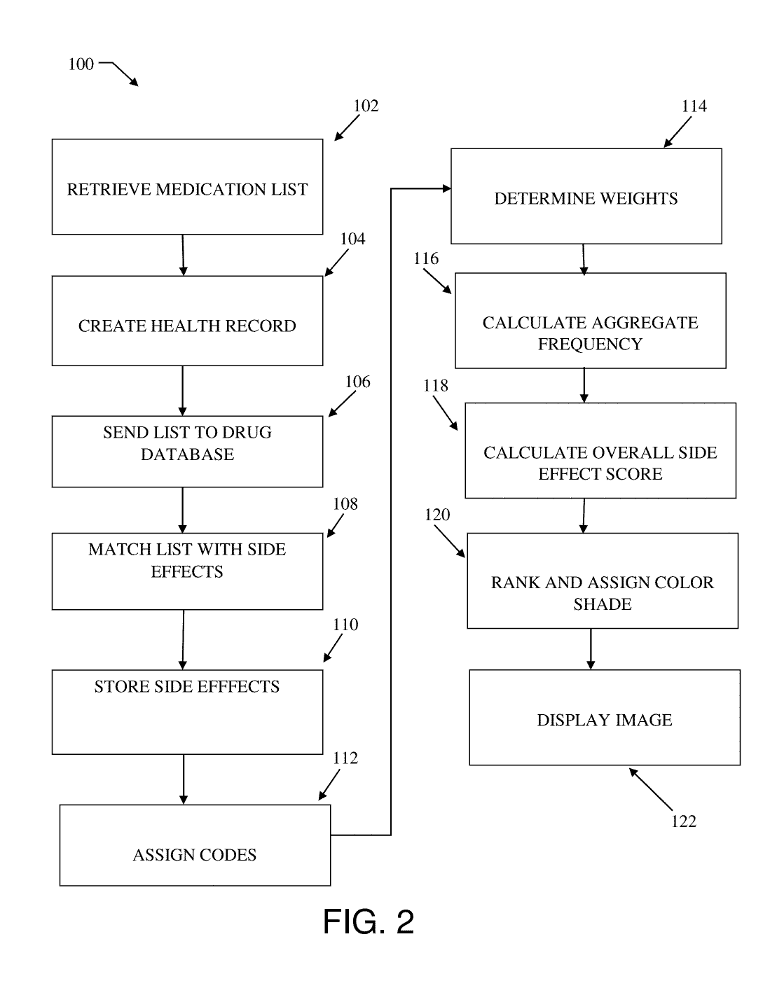

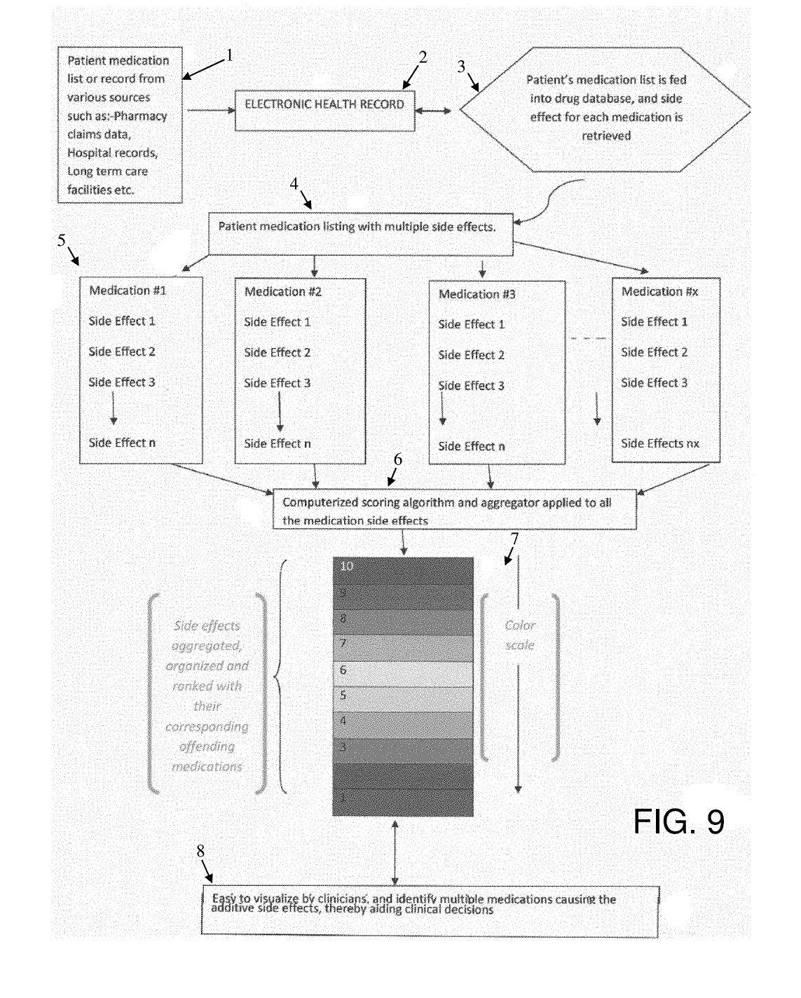

FIGS. 2 and 9 illustrate flow diagrams of the method 100 for organizing, prioritizing, and predicting the probability of medication adverse effects occurring using a scoring model analysis and algorithm based on prevalence and severity. While the methodology is described as being a series of acts or steps that are performed in a sequence, it is to be understood that the methodology is not limited by the order of the sequence. For instance, some acts or steps may occur in a different order than what is described herein. In addition, a step may occur concurrently with another step. Furthermore, in some instances, not all steps may be required to implement a methodology described herein.

Moreover, the steps or acts described herein may be computer-executable instructions that can be implemented by one or more processors and/or stored on a computer-readable medium or media. The computer-executable instructions may include a routine, a sub-routine, programs, a thread of execution, and/or the like. Still further, results of acts of the methodology may be stored in a computer-readable medium, displayed on a display device, and/or the like.

In step 102, the patient's medication list or record from the various sources is retrieved. This retrieving of this data may include entry of data by the user using the keyboard or other input device. In step 104, an electronic health record of the patient is created. The creation of this electronic health record may include entry of data by the user using the keyboard or other input device. In step 106, the patient's medication list is fed into the drug database. In step 108, the medication list is matched with a database of the side effects for each medication. In step 110, the side effects for each medication in the medication list is retrieved and stored in a database.

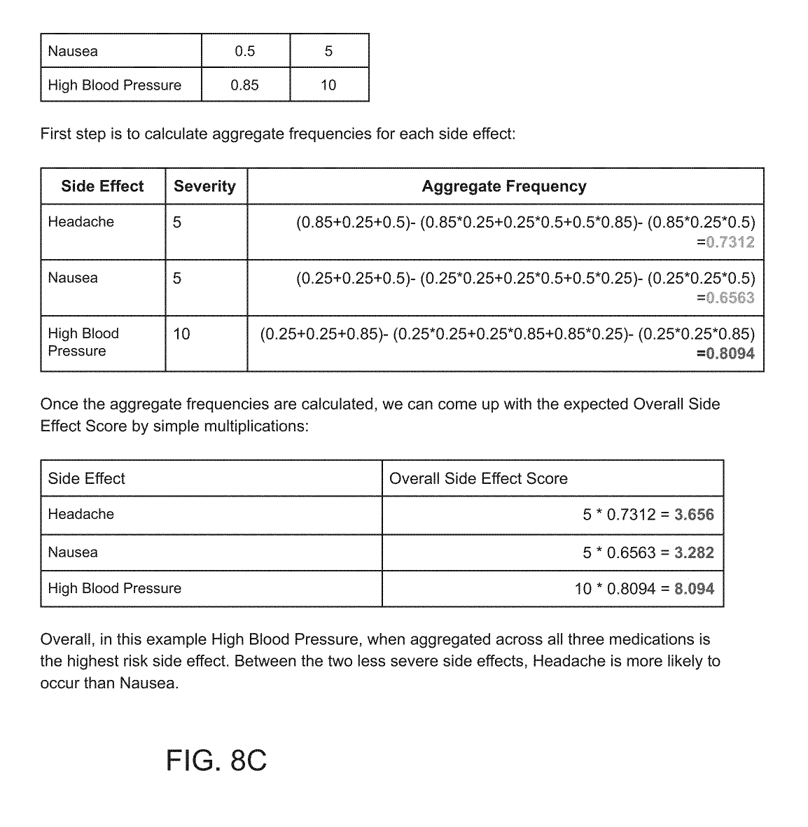

In step 112, the frequency of occurrence of each side effect associated with each medication is assigned a code and the severity of each side effect of each medication is assigned a code. In step 114, a weight is determined by the weighing applicator for the frequency of each side effect of each medication associated with the assigned code, and a weight is determined and assigned by the weight applicator for the severity of each side effect of each medication associated with the assigned code. Then, in step 116, the aggregate frequency of the frequencies of the each side effect for all of the medications is calculated by the aggregate frequency calculator. Then, in step 118, for each side effect, the overall side effect score is calculated by multiplying the aggregate frequency for that side effect by the severity weight for that side effect. To better illustrate steps 112 to 118, an example is shown in FIGS. 8A to 8C and discussed below.

In this example, suppose Patient A is taking three medications #1, #2, and #3 with side effects A, B, and C. Side effect A is a headache. Side effect B is Nausea. Side effect C is high blood pressure. The frequencies of the side effects are assigned a code and the weights of the frequencies are determined. The severity of the side effects are assigned a code and the weights of the severity are determined. The side effects with side effect frequencies and severities for this example is shown below.

| | Frequency | Severity |
|---|---|---|
| Medication #1 | | |
| Side Effect A | 0.85 | 5 |
| Side Effect B | 0.25 | 5 |
| Side Effect C | 0.25 | 10 |
| Medication #2 | | |
| Side Effect A | 0.25 | 5 |
| Side Effect B | 0.25 | 5 |
| Side Effect C | 0.25 | 10 |
| Medication #3 | | |
| Side Effect A | 0.5 | 5 |
| Side Effect B | 0.5 | 5 |
| Side Effect C | 0.85 | 10 |

The formula or method for calculating aggregate frequencies per side effect per severity level is shown in FIG. A and reproduced below.

$$Aggregate Frequency = {\sum }_{i} ( {Frequency}_{i} ) - {\sum }_{i , j} ( {Frequency}_{i} \times {Frequency}_{j} ) - {\sum }_{i , j , k} ( {Frequency}_{i} \times {Frequency}_{j} \times {Frequency}_{k} )$$

In the above formula I, j, and k range between 1 and 3 to indicate the medication. When calculating the product of frequencies, multiply all of the frequencies of side effects for medications where i≠j and i≠j≠k. The formula provides an aggregate frequency per side effect severity. So, for each side effect, this quantity needs to be calculated twice−once for low severity (5) and once for high severity (10). The results of the side effect severity aggregate Frequency are shown below.

Side Effect Severity Aggregate Frequency
Headache 5 (0.85+0.25+0.5)−(0.85*0.25+0.25*0.5+0.5*0.85)−(0.85*0.25*0.5)=0.7312
Nausea 5 (0.25+0.25+0.5)−(0.25*0.25+0.25*0.5+0.5*0.25)−(0.25*0.25*0.5)=0.6563
High Blood Pressure (0.25+0.25+0.85)−(0.25*0.25+0.25*0.85+0.85*0.25)−0.25*0.25*0.85)=0.8094

Then, the overall side effect score can be computed using the following formula:

Overall Side Effect Score=(Severity Weight)×(Aggregate Frequency).

The overall side effect score for each side effect in this example is listed below:

| Side Effect | Overall Side Effect Score |
|---|---|
| Side Effect A | 5 * 0.7312 = 3.656 |
| Side Effect B | 5 * 0.6563 = 3.282 |
| Side Effect C | 10 * 0.8094 = 8.094 |

Overall, in this example, high blood pressure, when aggregated across all three mediations is the highest risk side effect. Between the two less severe side effects, headache is more likely to occur than nausea.

In step 120, an electronic image is created showing the side effects ranked and assigned a color shade. In step 122, the electronic image is then displayed on the display showing the side effects with their rank and color shade in a manner that clinicians can easily visualize and identify multiple medications causing the side effects thereby aiding clinical decisions. For example, as seen at reference number 7 of FIG. 9, the electronic image may display a dark shaded brown rectangle with the number 10 in white located inside the rectangle to indicate a side effect score of 10 corresponding to a dark shaded brown color. The electronic image may display a light shaded brown rectangle with the number 7 in black located inside the rectangle to indicate a side effect score of 7 corresponding to a light shaded brown color. The electronic image may display a light shaded green rectangle with the number 6 in black located inside the rectangle to indicate a side effect score of 6 corresponding to a light shaded green color. The electronic image may display a dark shaded green rectangle with the number 1 in black located inside the rectangle to indicate a side effect score of 1 corresponding to a dark shaded green color. The electronic image or another electronic image may display a bar graph showing a bar in the color corresponding to the side effect score with the side effect displayed next to the bar. The printer may print the electronic image(s) shown on the display. The method may then recommend alternative drugs based on this display.

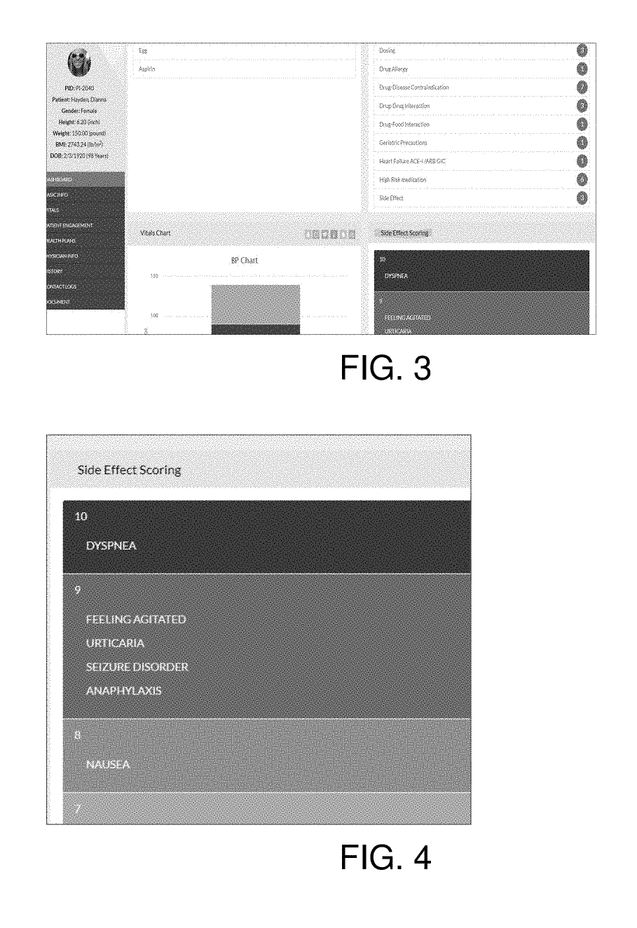

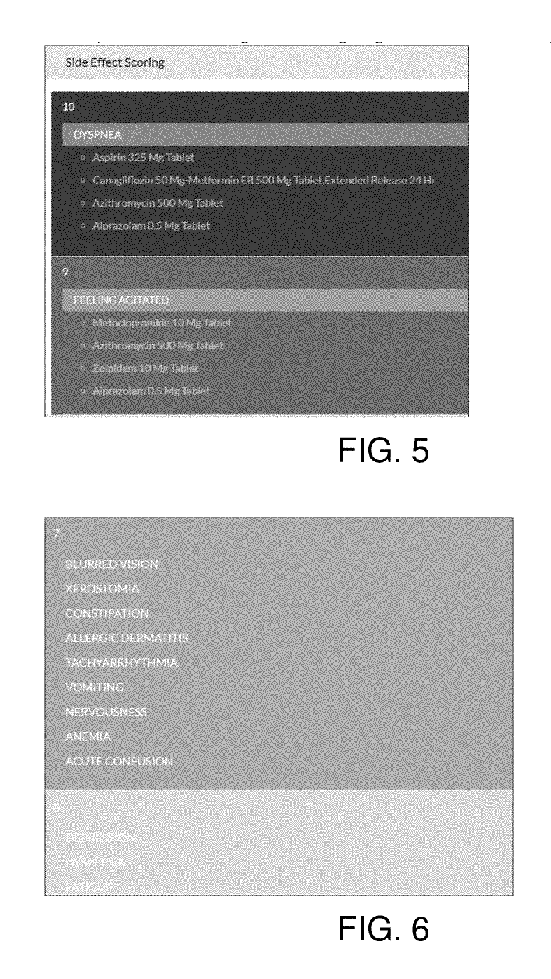

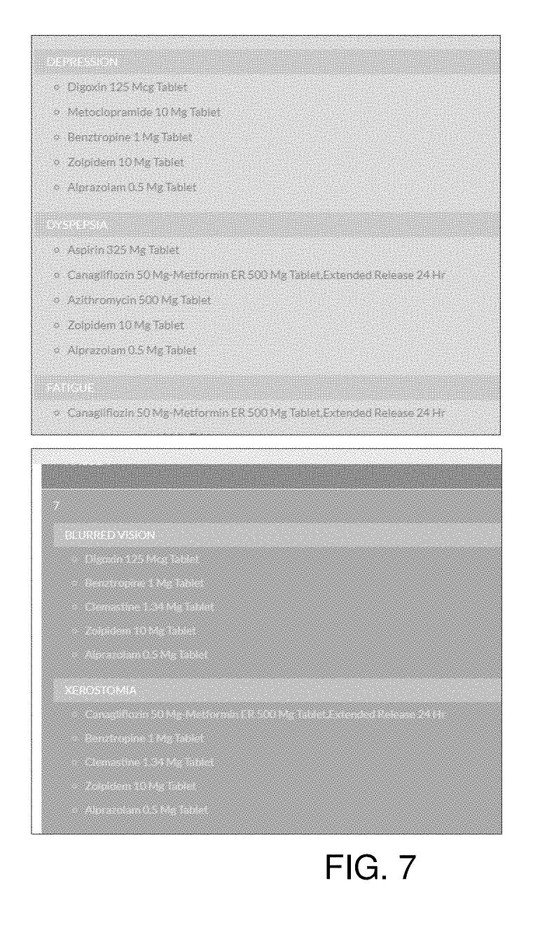

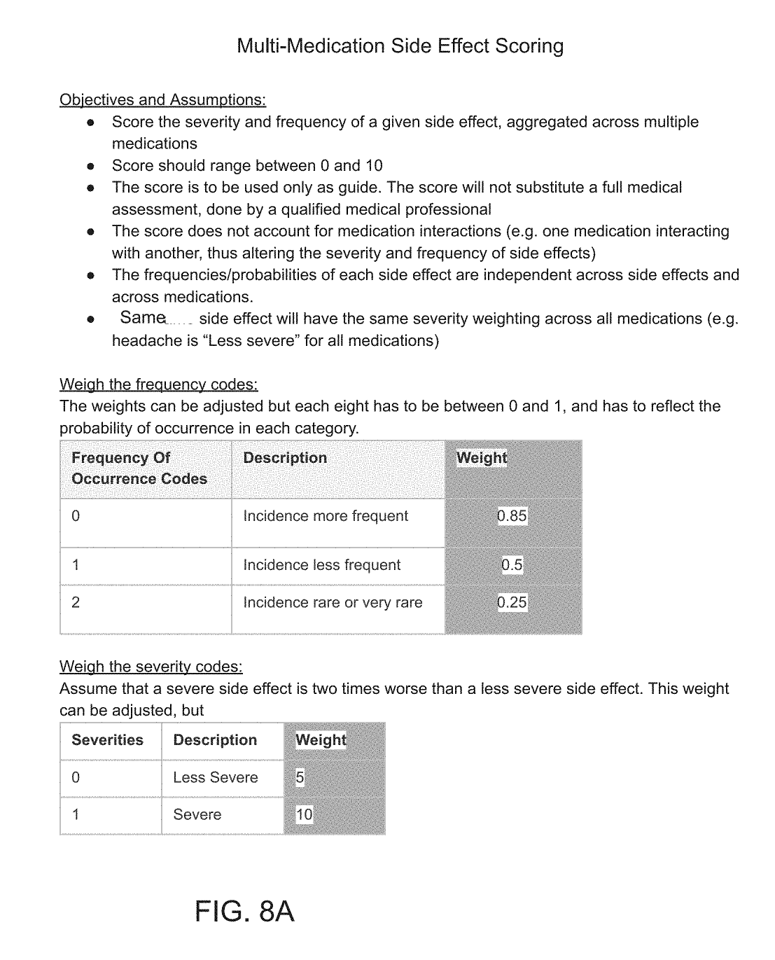

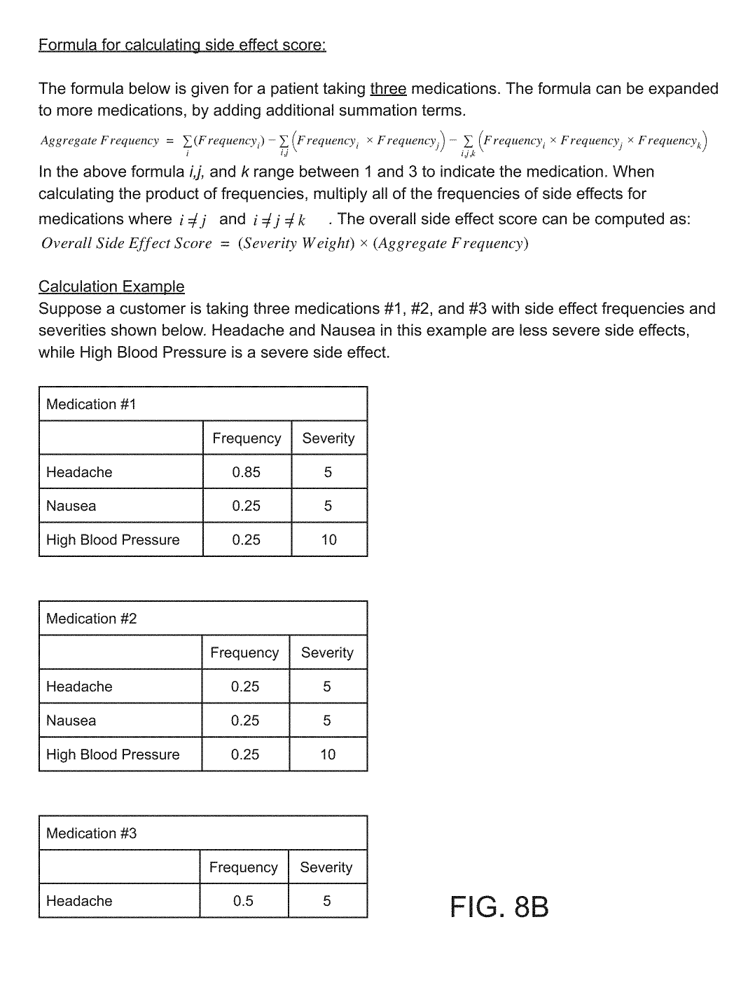

Other electronic images may be displayed. For example, FIG. 3 shows an electronic image of a patient profile dashboard showing the side effect scoring feature. FIG. 4 shows an electronic image of a portion of a customize side effect ranking with corresponding colors. FIG. 5 shows an expanded view of FIG. 4 showing the offending drugs for each side effect aggregate. FIG. 6 shows another view of an electronic image of a section of the customized side effect ranking with corresponding colors. FIG. 7 shows an expanded view of an electronic image showing the offending drugs and the corresponding side effects.

FIGS. 8A-8C and 9 further illustrate the system and method for depicting medication adverse effects of a patient, and scoring analysis based on prevalence and severity, patient's medication list for use in predicting additive side effects (FIGS. 8A to 8C), which utilizes a clinician such as a pharmacist, physician, counselor, technician, nurse, physician assistant in association with a computer 6 (FIG. 9). The patient's medication list from the pharmacy claims data, the hospital record and other sources 1 (FIG. 9) is received and transmitted to a clinician through an Electronic Health Record 2 (FIG. 9) for ranking or predicting the potential side effects that are most likely to occur in a patient. This helps the clinician to focus on the agents or drugs within the patient's' medication list likely to cause additive side effects to the patient as seen in block 4 of FIG. 9.

This algorithm would be embedded or integrated into a medication management software or Electronic Health Record (EHR) 2 (FIG. 9) used by a clinician to streamline and customize medication adverse effects to enable clinicians to identify and focus on side effects which are likely to occur in their patients. The system (FIG. 9) may comprise a Patient's Medication List (PML) uploaded via EHR interface or inputted manually into the system by the Clinician that includes, for each patient, a list of their medications as prescribed by their healthcare providers and also over the counter medications (OTCs) including herbals, vitamins and supplements. The system matches the patient's medication list with the side effects or adverse effects data provided by FDA through its Drug database 3 (FIG. 9). Based on the information obtained by the clinician from matching the patient's' medication list with FDA Drug database, the system streamlines, aggregates and condenses all the potential side effects of all the drugs in the patient's list as illustrated by blocks 4 and 5 of FIG. 9, while excluding allergic reactions as performed at block 6 of FIG. 9. The system creates a customized Medication adverse effects panel which is represented and organized on a color scale 7 (FIG. 9), and on a numerical scale of 1-10 as also illustrated by reference number 7 of FIG. 9. This method is easy to visualize by the clinician, and helps the clinician to conduct a predictive medication risk analysis 8 (FIG. 9).The system uses an algorithm to streamline and predict potential side effects (FIGS. 8 to 8C).

Thus, this system and method uses a scoring algorithm based on prevalence and severity to aggregate all the patient's side effects, prioritizing them on a color scale, and making it easier for clinicians such as a pharmacists, physicians, counselors, technicians, nurses, physician assistants, to easily visualize in order to help:
1. Predict medication adverse effects that would likely be experienced by the patient based on their medication regimen
2. De-prescribe or dose-adjust offending medications
3. Diagnose the etiology of “idiopathic” conditions being experienced by the patient which is likely caused by multiple medications
4. Easily identifies and groups/organizes offending medications together
5. Makes it easier for clinicians to educate patients on potential side effects.

The application will then suggest customized therapeutic alternatives to the offending medication based on these criteria: 1) individual patient's chronic conditions and 2) the suggested alternatives won't cause the same side effects. This would enable clinicians to come up with certain solutions aimed at de-prescribing or eliminating the offending medications, or educating the patient on potential adverse effects and then easily and efficiently replacing the offending medications with therapeutic alternatives without a tedious and extensive research, thereby saving the clinicians time.

The system and method helps clinicians streamline, aggregate, score, visualize, organize and predict a patient's adverse effects in order to develop the correct intervention for the addressing medication related problems.

The system and method provides information which complies with the legislation, and which helps predict additive side effects of all the medication in a patient's cabinet for easier treatment of patients who have certain risk factors or other high-risk problems.

This system and method provides a “customize side-effect message” that the clinician can access on the user device that compiles patient's adverse effects and scores them based on prevalence and severity using an interactive telecommunications system that helps the clinician come up with certain solutions, which is aimed at de-prescribing or eliminating the offending medications, or helping the patient implement other healthy behavioral changes based upon their medication history over a period of time.

This system and method enables clinicians to prioritize and predict the severity and frequency of a given side effect on a patient, and aggregating the score across multiple medications in the patient's cabinet. This system and method weighs the frequency of each side effect on a given patient's medication using frequency codes that vary from 0-2 with 0 representing “more frequent” and 2 representing “incidence rare or very rare.”.

This system and method helps the clinician to determine certain treatment options based on severity codes, and also guide him/her in reaching a decision as to whether a particular side effect is more frequent or less frequent. This system and method provide a formula that provides an aggregate frequency per side effect severity. So, for each side effect, the Clinician will easily calculate the severity using a simple formula. Thus, a clinician is able to make his treatment decision based on a simple computation that takes into account the number of medications per patient and factors in the side effect frequencies per patient, and scores the severity based on this model.

This application relates to the general field of a scoring system and method in patient's medication management, using a computerized database that condenses, aggregates and organizes adverse effects in groups based on prevalence and severity while listing the offending drugs and side effects on a color scale. The application will then suggest customized therapeutic alternatives to the offending medication based on these criteria: 1) individual patient's chronic conditions and 2) the suggested alternatives won't cause the same side effects. This would enable clinicians to come up with certain solutions aimed at de-prescribing or eliminating the offending medications, or educating the patient on potential adverse effects and then easily and efficiently replacing the offending medications with therapeutic alternatives without a tedious and extensive research, thereby saving the clinicians time.

Although various embodiments of the disclosed system and method have been shown and described, modifications may occur to those skilled in the art upon reading the specification. The present application includes such modifications and is limited only by the scope of the claims.

What is claimed is:

1. A method comprising:
a) creating an electronic health record of a patient based on a medication list of the patient;
b) storing the electronic health record of the patient into a first database;
c) matching the medication list in the electronic health record with a second database of side effects data for each medication in the medication list;
d) storing the side effects data for each medication in a third database, wherein the side effects data includes a frequency of occurrence for each side effect associated with each medication, wherein the side effects data includes a severity of each side effect associated with each medication;
e) determining the overall side effect score based on the aggregate frequency for that side effect and the severity weight for that side effect;
f) creating an electronic image that shows the side effects ranked and assigned a shade of color; and
g) displaying the electronic image on a display.

2. The method of claim 1, wherein displaying the electronic image on a display includes displaying a dark shaded rectangle with a lighter colored number located inside the rectangle to indicate a side effect score of that number corresponding to the dark shaded color.

3. The method of claim 1, wherein displaying the electronic image on a display includes displaying a bar graph showing a bar in the color corresponding to the side effect score with the side effect displayed next to the bar.

4. The method of claim 1 further comprising printing the electronic image displayed on the display.

5. The method of claim 1 further comprising displaying recommended alternative drugs on the display based on the electronic image.

6. The method of claim 1 wherein displaying the electronic image on a display includes displaying an electronic image of a patient profile dashboard showing the side effect score.

7. The method of claim 1, wherein determining the overall side effect score based on the aggregate frequency for that side effect and the severity weight for that side effect comprises:
determining the weight for the frequency of each occurrence of each side effect of each medication associated with the assigned code;
determining the weight for the severity of each side effect of each medication associated with the assigned code;
determining the aggregate frequency of the frequencies of each side effect for all of the medications; and
multiplying the aggregate frequency for that side effect by the severity weight for that side effect.

8. The method of claim 7, wherein determining the aggregate frequency of the frequencies of each side effect for all of the medications is performed by
$$Aggregate Frequency = {\sum }_{i} ( {Frequency}_{i} ) - {\sum }_{i , j} ( {Frequency}_{i} \times {Frequency}_{j} ) - {\sum }_{i , j , k} ( {Frequency}_{i} \times {Frequency}_{j} \times {Frequency}_{k} )$$
where i, j, and k range between 1 and 3.

9. The method of claim 1, wherein determining the overall side effect score based on the aggregate frequency for that side effect and the severity weight for that side effect further comprises:
assigning a code to the frequency of occurrence for each side effect;
assigning a code to the severity of each side of each medication;
determining the weight for the frequency of each occurrence of each side effect of each medication associated with the assigned code;
determining the weight for the severity of each side effect of each medication associated with the assigned code;
determining the aggregate frequency of the frequencies of each side effect for all of the medications; and
multiplying the aggregate frequency for that side effect by the severity weight for that side effect.
